(19) United States
(12) Patent Application Publication
(10) Pub. No.: US 2003/0069698 A1
(43) Pub. Date: Apr. 10, 2003

(54) Method and system for predicting pharmacokinetic properties

(76) Inventors: Mamoru Uchiyama, Aichi-ken (JP); Kazunari Hattori, Aichi-ken (JP); Kaoru Shimada, Aichi-ken (JP)

Correspondence Address: Gregg C. Benson, Pfizer Inc., Patent Department, MS 4159, Eastern Point Road, Groton, CT, 06340, US

(21) Appl. No.: 09/876,767

(22) Filed: Jun. 7, 2001

Related U.S. Application Data
(60) Provisional application No. 60/211,864, filed on Jun. 14, 2000.

(51) Int. Cl. G06F 19/00; G01N 33/48; G01N 33/50

(52) U.S. Cl. 702/19; 702/27

(57) ABSTRACT

This invention provides a method for predicting pharmacokinetic properties of molecules comprising the steps of:

(a) preparing 2D-structures of molecules used as a training set;

(b) constructing a 2D-fingerprint by counting the number of structural descriptors that potentially relate to a pharmacokinetic property, either manually or automatically using internally developed macro; wherein said structural descriptors consist of predefined 20 to 80 atoms/fragments or substructures;

(c) analyzing the obtained 2D-fingerprint by a statistical analysis method to correlate with the pharmacokinetic property of the molecule to yield a quantitative structure-property relationship (QSPR) model; and

(d) calculating the pharmacokinetic property of a trial molecule using the above obtained QSPR model.

A system for this invention is also provided. According to this method and system, it is possible to predict pharmacokinetic properties of molecules prior to synthesis, without labor-intensive and time-consuming experimentation.

### CROSS REFERENCE TO RELATED APPLICATION

This application claims the benefit of U.S. Provisional Application No. 60/211,864 filed Jun. 14, 2000.

### TECHNICAL FIELD

This invention relates to a method and system to predict pharmacokinetic (ADME) properties such as drug absorption (permeability), distribution, metabolism, and excretion, which are crucial properties in drug discovery.

### BACKGROUND ART

Experimental measurements to obtain pharmacokinetic properties are time-consuming and labor-intensive. Moreover experiments require a significant amount of actual compounds. Thus, the computational methods to predict such properties of virtual compounds are highly desirable in prioritization of targets prior to synthesis.

So far, similar descriptors as conventionally employed in the quantitative structure activity relationship (QSAR) analysis (steric bulk, lipophilicity, HOMO energy, etc.) have been adopted in quantitative structure property relationship (QSPR) analysis to correlate with PK-related parameters (t½, clearance, or oxidation rate, etc.) (Lien, E. J. et al. Acta Pharm. Jugosl. 1984, 34, 123-131; Baeaernhielm, C. et al. Chem.-Biol. Interact. 1986, 58, 277-288). Graph theory derived parameters (molecular connectivity indexes, etc.) have been also used for this purpose (Markin, R. S. et al. Pharm. Res. 1988, 5, 201-208; Garcia-March, F. J. et al. J. Pharm. Pharmacol. 1995, 47, 232-236). Recently reported QSPR methods necessitate calculation on 3D-structures that is still computationally intensive (Lombardo, F. et al. J. Med. Chem. 1996, 39, 47504755; Palm, K. et al. J. Med. Chem. 1998, 41, 5382-5392; Clark, D. E. J. Pharm. Sci. 1999, 88, 815-821). The QSPR methods also necessitate complete set of molecular parameters (van de Waterbeemd, H. et al. Quant. Struct.-Act. Relat. 1996, 15, 480-490) that require experimental measurements to be determined.

2D-fingerprints are frequently employed in molecular similarity/diversity analysis (e.g. ISIS™/Base similarity search or SYBYL™/Selector), high-volume QSAR analysis (e.g. HQSAR, vide infra), and other drug discovery scenes. To date there has been no report on development of 2D-fingerprints descriptors to analyze pharmacokinetic properties.

WO 98/07107 discloses a MOLECULAR HOLO(GRAM QSAR (HQSAR™) to develop high volume QSAR models. HQSAR™ uses molecular hologram based on fragments counts to deal with mostly potency/activity. A symposium proceeding (Niwa, T. “Prediction of Human Intestinal Absorption of Drug Based on Neural Network Modeling”; 27^{th} Symposium on Structure-Activity Relationships held in Japan, Nov. 10, 1999) describes a method to estimate human intestinal absorption (HIA) based on molecular topological indexes derived from 2D-structure.

It could be highly desirable to provide a system and method to predict pharmacokinetic properties of actual and virtual molecules with high performance (predictivity and speed) and wide applicability to diverse molecules.

### BRIEF DISCLOSURE OF THE INVENTION

This invention provides a new method and system for QSPR analysis and prediction based on only 2D-structure that allows us to predict hundreds of compounds rapidly. The method and system of this invention employs 2D-fingerprints, an array of the counts of functional groups as descriptors for QSPR.

This invention provides a method for predicting pharmacokinetic properties of molecules comprising the steps of:

(a) preparing 2D-structures of molecules used as a training set;

(b) constructing a 2D-fingerprint by counting the number of structural descriptors that potentially relate to a pharmacokinetic property, either manually or automatically using internally developed macro; wherein said structural descriptors consist of predefined 20 to 80 atoms/fragments or substructures;

(c) analyzing the obtained 2D-fingerprint by a statistical analysis method to correlate with the pharmacokinetic property of the molecule to yield a quantitative structure-property relationship (QSPR) model; and

(d) calculating the pharmacokinetic property of a trial molecule using the above obtained QSPR model.

This invention also provides a system for predicting pharmacokinetic properties of molecules comprising:

(a) means for preparing 2D-structures of molecules used as a training set;

(b) means for constructing a 2D-fingerprint by counting the number of structural descriptors that potentially relate to a pharmacokinetic property, wherein said structural descriptors consist of predefined 20 to 80 atoms/fragments or substructures;

(c) means for analyzing the obtained 2D-fingerprint by a statistical analysis method to correlate with the pharmacokinetic property of the molecule to yield a quantitative structure-property relationship (QSPR) model; and

(d) means for calculating the pharmacokinetic property of a trial molecule using the above obtained QSPR model.

Another aspect of this invention provides a method wherein the pharmacokinetic property is absorption.

Another aspect of this invention provides a method wherein the pharmacokinetic property is distribution.

Another aspect of this invention provides a method wherein the pharmacokinetic property is metabolism

Another aspect of this invention provides a method wherein the pharmacokinetic property is excretion.

Another aspect of this invention provides a method wherein the internally developed macro comprises the macro script 2dfp.spl or 2dfp_abs.spl, written in SYBYL™ Programming Language (SPL).

Preferably, each of the steps of the methods of the invention is carried out using molecular modeling software, databases or drawing software. More preferably one is such as SYBYL™, version 6.5 (Tripos Inc., St. Louis, Mo.). The database includes, for example, ISIS Base™ version 2.2.1 (MDL information Systems, Inc. San Leandro, Calif.). The drawing software includes such as SYBYL™/SKETCH option, ISIS™ Draw version 2.2.1, Chem Draw Pro™ version 5.0 (CambridgeSoft. Corp. Cambridge, Mass.) and SMILES™ (Daylight Chemical Information Systems, Inc., Mission Viejo, Calif.). Other modeling software, databases, and drawing software known to those of skill in the art can also be used.

This invention enables us to perform virtual screening for synthetic targets and data mining using databases as well as drug design to optimize the pharmacokinetic profiles. Based on the QSPR model in this invention, it is possible to predict pharamacokinetic properties of molecules prior to synthesis, without labor-intensive and time-consuming experiment. This invention relies on 2D-fingerprint modeling requiring only 2D-structure, which enables us to perform rapid calculation to predict hundreds of compounds without tedious calculation about 3D-structure. Moreover, 2D-fingerprint used in this invention comprises only 20-80 bits.

### DESCRIPTION OF FIGURES

FIG. 1 is a flowchart showing the overall process of the invention.

FIG. 2 shows a plot of actual vs. calculated log t½.

FIG. 3 shows a plot of actual vs. calculated log(Pₐₚₚ* 10⁶).

FIG. 4 shows a plot of actual vs. calculated logBB.

### DETAILED DISCLOSURE OF THE INVENTION

The term “molecules used as a training set” as used herein, refers to the molecules whose pharmacokinetic properties have been already determined experimentally and used to develop a predictive QSPR model.

The term “pharmacokinetic properties” as used herein, refers to the properties of molecules related to metabolism, absorption (permeability), distribution, and excretion (ADME).

A number of experimental methods or models are known in ADME.

Examples of absorption studies are 1) kinetic studies based on measuring plasma concentration, urinary fecal excretions and gastrointestinal disposition after oral administration in vivo, 2) single-pass perfusion method, recirculation method, loop method in situ, and 3) everted sacs method, methods of using brush border membrane vesicles, isolated cells, and cultured cells (Caco-2) in vitro and the like.

Examples of distribution studies are 1) the method of measuring concentration of target organs after administration by various technique such as HPLC, LC-MS, autoradiography and microdialysis in vivo, 2) brain perfusion methods such as vascular reference method (brain uptake index) in situ, and 3) methods of using isolated cells or cultured cells (such as endothelial cell) in vitro and the like.

Examples of metabolism studies are 1) kinetic studies based on measuring concentrations of drugs and the metabolites after adequate administration routes such as intravenous administration, administration per portal vain in vivo, and in situ, 2) kinetic studies such as a half-life of drugs in mammalian organ (liver, kidney, intestine, etc. with slices, homogenates and microsomes etc) and in isolated cells or cultured cells such as hepatocytes in vitro and the like.

Examples of excretion studies are 1) kinetic studies based on measuring concentration of drugs in urine, bile, feces etc after administration in vivo, 2) enzymatic studies of excretion via pumps such as P-glycoprotein, in vitro and the like.

The term “2D-fingerprint” as used herein, refers to a 2D-molecular measure in which a bit in a data string is set corresponding to atoms/fragments or substructures.

The term “predefined atoms/fragments or substructures” as used herein, refers to atoms or functional groups relating to a phrmacokinetic property, which are based on the literature source (Bonse, V. G., Metzler, M. “Biotransformationen Organischer Fremdsubstanzen” (Yakubutu-Taisha) in Japanese Asakura, Tokyo (1980); Kato, R., Kamatani, T. “Yakubutu-Taishagaku” in Japanese, Tokyo-Kagaku-Dojin, Tokyo, chapter 4, 93-123 ,(1995)), otherwise refers to functional groups such as saturated or unsaturated bonds, rings (aromatic or cycloalkyl), amines, anilines, nittrogen in aromatics, imines/nitriles/guanidine/amidine, oxyamine(N—O)/nitro/azo-/hydrazin, amide/thioamide/sulfonamide/, alcohollether/aldehyde/ketone/ester/carboxylic acid/carbothioic acid/sulfinic acid/sulfonic acid, halogen, oxygen or sulfur functional groups, and total number of carbon, hydrogen, nitrogen, oxygen, sulfur or phosphorus atom.

The term “internally developed macro” as used herein, refers to an internally developed Sybyl Programming Language (SPL) code. Preferable internally developed macro is as described in Working Examples 4 and 5.

The QSPR model based on 2D-fingerprints for metabolism predicts half-life of molecules in a human liver microsome mixture with stood predictivity. The 2D-fingerprints for absorption are successfully employed to develop a higly predictive QSPR model on drug permeability across monolayer Caco-2 cells. Similarly the present 2D-fingerprints/PLS modeling can be applied to develop statistically significant QSPR models on blood-brain barrier partitioning of the structurally diverse set. Thus, the method of this invention requiring only 2D-structures of the pertinent molecules enables to perform virtual screening of synthetic targets and data mining using molecular database as well as drug design to optimize the pharmacokinetic profiles.

FIG. 1 illustrates the method of this invention. This invention will be described in more detail with reference to FIG. 1. Computational modeling studies can be carried out using molecular modeling software, preferably SYBYL™ on a Silicon Graphics Octane™ workstation. The method of this invention comprises the following steps:

(a) 2D-structure of a molecule can be prepared by retrieving from a database such as ISIS™/Base, or by constructing manually with drawing software. The drawing software includes, for example, SYBYL™/SKETCH option (on the workstation), or ISIS™ Draw, Chem Draw™ and SMILES™ on (PC such as Windows NT client PC). The 2D-structure thus prepared can be transferred to the workstation, and stored in the molecular database.

(b) The prepared 2D-structure of a molecule can be imported into molecular modeling software such as SYBYL™ as a MOL2 format. 2D-fingerprints can be constructed by the use of internally developed macro script 2dfp.spl or 2dfp_abs.spl, written in SYBYL™ Programming Language (SPL) implemented in SYBYL™, or by manually counting the number of the atoms/fragments or substructures. The macro program converts 2D-structures stored in the molecular database as a MOL2 format into a SYBYL™ line notation (SLN) format. Subsequently, the macro searches each SLN for the substructures potentially related to a pharmacokinetic property that match the queries described in the macro (as shown in Working Example 4), wherein the queries are predefined as the substructures (20 to 80 atoms/fragments). Finally the macro enumerates the substructure counts, and records them as 2D-fingerprints.

(c) Statistical analysis is performed to obtain a correlation between the obtained 2D-fingerprints and the pharmacokinetic property. Any analytical method such as partial least square (PLS) algorithm, sample-distance partial least squares (SAMPLS; Bush, B. L. et al. J. Computer-Aided Mol. Design, 1993, 7, 587-619), genetic algorithm or neural network can be employed to yield an optimal quantitative structure property relationship (QSPR) model.

(d) The pharmacokinetic property for trial molecules can be calculated based on the above obtained QSPR model.

The pharmacokinetic properties of the molecule such as absorption, distribution, metabolism and excretion, can be apparent permeability coefficients (Pₐₚₚ) [cm/sec], blood-brain barrier partitioning ratio {(C_{brain}/C_{blood})=BB}, half-life(T_{½}) in mammalian liver microsome and the like.

The system of this invention can be constructed using appropriate computer hardware such as a Silicon Graphics Octane™ workstation and software as described above.

This invention will be further described below with reference to the following Working Examples.

### EXAMPLES

### Example 1

### Development and Validation of QSPR for Half Life in Human Liver Microsome

Computational modeling studies were carried out using a Silicon Graphics Octane™ workstation. A congeneric series of 54 compounds of Formula (I) (as shown in the following Table 1.) with a variety of substituent groups were used as a training set for analysis.

TABLE 1

| | |
|---|---|
| | (I) |
| 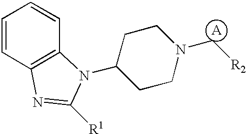 | |

| # | A | R1 | R2 |
|---|---|---|---|
| 1a | cycloheptyl | piperidinyl | Ph |
| 2a | cycloheptyl | H₂N(CH₂)₂O— | Ph |
| 3a | cycloheptyl | 4-aminopiperidyl | Ph |
| 4a | cycloheptyl | H₂N(CH₂)₂C(O)— | Ph |
| 5a | cycloheptyl | H₂N(CH₂)₂CONH— | Ph |
| 6a | cyclohepten-1-yl | 4-aminopiperidyl | Ph |
| 7a | cyclooctyl | H₂NCH₂CONH— | Ph |
| 8a | cycloheptyl | H₂N(CH₂)₃— | Ph |
| 9a | cycloheptyl | 4-aminocyclohexylamino | Ph |
| 10a | cyclohepten-1-yl | piperazinyl | Ph |
| 11a | cycloheptyl | piperazinyl | Ph |
| 12a | cycloheptyl | H₂N(CH₂)₂NH— | Ph |
| 13a | cycloheptyl | H₂NC(CH₃)₂CH₂NH— | Ph |
| 14a | cycloheptyl | N-methylpiperazinyl | Ph |
| 15a | cycloheptyl | piperidinylamino | Ph |
| 16a | cycloheptyl | 4-aminopiperidyl | CH₃ |
| 17a | cycloheptyl | piperidinyl | CH₃ |
| 18a | cycloheptyl | H₂N(CH₂)₁₀NH— | Ph |
| 19a | cycloheptyl | 4-aminoazetidinyl | Ph |
| 20a | cycloheptyl | H₂N(CH₂)₈NH— | Ph |
| 21a | cycloheptyl | (CH₃)₂N(CH₂)₂NH— | Ph |
| 22a | cyclooctyl | N-methylpiperazinyl | Ph |
| 23a | cycloheptyl | piperazinyl | isopropyl |
| 24a | cycloheptyl | piperidinecarboximidamide | Ph |
| 25a | cycloheptyl | H₂N(CH₂)₆NH— | Ph |
| 26a | cycloheptyl | H₂N(CH₂)₄NH— | Ph |
| 27a | cyclononyl | amino | Ph |
| 28a | cycloheptyl | CH₃NH(CH₂)₂NH— | Ph |
| 29a | cyclooctyl | piperazinyl | CH₃ |
| 30a | cycloheptyl | 4-aminopiperidyl | vinyl |
| 31a | cycloheptyl | isopropyl | Ph |
| 32a | cycloheptyl | 2-guanidinoethyl | Ph |
| 33a | cycloheptyl | mathanesulfoonyl | Ph |
| 34a | cycloheptyl | piperidinyloxy | Ph |
| 35a | cycloheptyl | dimethylamino | Ph |
| 36a | cycloheptyl | amino | Ph |
| 37a | cycloheptyl | CH₃CONH— | Ph |
| 38a | cycloheptyl | hydroxypiperidinyl | Ph |
| 39a | cycloheptyl | H₂N(CH₂)₃SO₂— | Ph |
| 40a | cycloheptyl | methylamino | Ph |
| 41a | cycloheptyl | methyl | Ph |
| 42a | cyclooctyl | piperazinyl | CH₃ |
| 43a | cycloheptyl | isobutyl(NH₂)CHCONH— | Ph |
| 44a | cycloheptyl | methylamino | CH₃ |
| 45a | cycloheptyl | methoxy | Ph |
| 46a | cyclooctyl | methylamino | normalpropyl |
| 47a | cyclooctyl | methylamino | CH₃ |
| 48a | cyclooctyl | methylpiperazinyl | CH₃ |
| 49a | cycloheptyl | H | Ph |
| 50a | cyclononyl | methylamino | CH₃ |
| 51a | cyclononyl | methylpiperazinyl | CH₃ |
| 52a | cycloheptyl | isobutyl(NH₂)CHCONH— | CH₃ |
| 53a | cycloheptyl | H₂N(CH₂)₂CONH— | Ph |
| 54a | cycloheptyl | H₂N(CH₃)₂CCONH— | Ph |

Half-life (t½) in vitro for each compound was determined by HPLC analysis of the reaction mixture with human liver microsome. The employed 2D-structures were retrieved from ISIS™/Base (version 2.2.1, MDL Information Systems, Inc., San Leandro, Calif.) or constructed with ISIS™/Draw (version 2.2.1, MDL Information Systems, Inc., San Leandro, Calif.) on a WinNT client PC, followed by being transferred to the Octane workstation and stored in a molecular database. The 2D-fingerprints were constructed by use of a newly developed macro script 2dfp.spl, written in SYBYL™ Programming Language (SPL), which was implemented in SYBYL™ (version 6.5, Tripos Inc., St. Louis, Mo.). The macro program converted 2D-structures stored in the molecular database as MOL or MOL2 format into SYBYL™ line notation (SLN) format, and counted the number of the atoms or functional groups that matched queries defined in a table described in the macro program. The atoms or functional groups susceptible to be involved in metabolism were assigned on the basis of the literature source (Bonse, V. G., Metzler, M. “Biotransformationen Organischer Fremdsubstanzen” (Yakubutu-Taisya, in Japanese) Asakura, Tokyo (1980); Kato, R.; Kamataki, T. “Yakubutu-Taisyagaku” in Japanese, Tokyo-Kagaku-Dojin Tokyo (1995)). Partial least square (PLS) algorithm in QSPR module in SYBYL™ was employed to correlate the aforementioned 2D-fingerprints and t½ to produce QSPR model. Thirty-eight bits out of whole 2D-fingerprints used since 25 bits with all the same value or 0 were dropped. SAMPLS run in crossvalidation step (leave-1-out) identified the optimum PLS component as 5 (N=54, Std. Error_prediction=0.414; q²=0.518). Non-crossvalidation PLS analysis resulted in a significant five-component model with the following statistics: Std. Error_Est.=0.219, r²=0.865, F(n1=5, n2=48)=61.3.

FIG. 2 shows the plot of actual vs. calculated log t½ (closed circles). For validation of the present QSPR model, the prediction of half-life for the test set (12 compounds) was performed. As indicated open squares in FIG. 2, the model has a fairly good predictivity, which allows us to prioritize the targets for synthesis.

### Example 2

### Development of QSPR for Caco-2 Permeability

Unless otherwise noted similar computational molecular modeling were performed as described in Example 1. Table 2 enlists 21 structurally diverse compounds as a training set, whose apparent permeability coefficients (Pₐₚₚ) [cm/sec] of a compound across Caco-2 cells was used as in literature source (Yee, S. Pharm. Res. 1997, 14, 763-766). The counts of substructures to match with the predefined queries were encoded as a array of integers by a similar SPL script (2dfp_abs.spl) to afford 2D-fingerprints as descriptors employed in the correlation analysis. SAMPLS run in crossvalidation step (leave-1-out) identified the optimum PLS component as 2 (N=21, Std. Error_prediction=0.444; q²=0.463). Non-crossvalidation PLS analysis resulted in a significant two-component model with the following statistics: Std. Error_Est.=0.254, r²=0.824, F(n1=2, n2=18)=42.1. FIG. 3 shows the plot of actual vs. calculated log(Pₐₚₚ* 10⁶)

TABLE 2
Training set compounds with apparent permeability.

| | Pₐₚₚ * 10⁶ | | Pₐₚₚ * 10⁶ | | Pₐₚₚ * 10⁶ |
|---|---|---|---|---|---|
| Compd. | (cm/sec) | Compd. | (cm/sec) | Compd. | (cm/sec) |
| Azithro- | 1.04 | Diazepam | 70.97 | Prazosin | 43.60 |
| mycin | | | | | |
| Benzyl- | 1.96 | Erythro- | 1.80 | Propranolol | 27.50 |
| penicillins | | mycin | | | |
| Caffeine | 50.50 | Flucona- | 29.80 | Quinidine | 20.40 |
| | | zole | | | |
| Chloram- | 20.60 | Ibuprofen | 52.50 | Tenidap | 51.20 |
| phenicol | | | | | |
| Clonidine | 30.10 | Imi- | 14.10 | Testosterone | 72.27 |
| | | pramine | | | |
| Desi- | 21.60 | Metho- | 1.20 | Trovaflox- | 30.23 |
| pramine | | trexate | | acin | |
| Dexa- | 23.40 | Naloxone | 28.20 | Ziprasidone | 12.30 |
| methasone | | | | | |

### Example 3

### Development of QSPR for Blood-Brain Barrier Partition

Unless otherwise noted, similar molecular modeling was performed as described in Example 1. Blood-brain barrier partitioning ratio, {log(C_{brain}/C_{blood})=logBB } for “drug-like” compounds (N=35, Chart 1) as a training set were used as in literature source (Lombardo, F. et al., J. Med. Chem. 1996, 39, 4750-4755.). The 2D-fingerprints were calculated as above example. PLS modeling to correlate 2D-fingerprints with BBB partitioning ratio showed the following statistics. Crossvalidation (SAMPLS, leave-1-out): the optimum PLS component=3, N=35, Std. Error_prediction=0.69; q²=0.29. Non-crossvalidation: Std. Error_Est.=0.38, r²=0.78, F₍3,31)=37 4.

CHART 1
Compounds employed in the analysis. (compound 36 for validation)

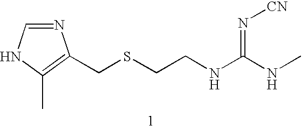

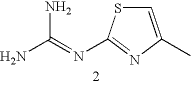

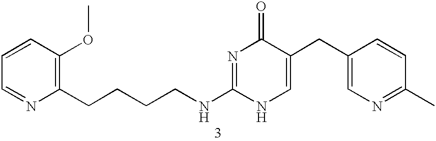

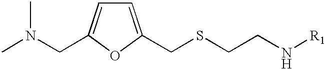

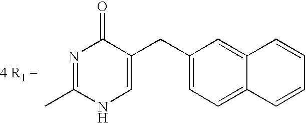

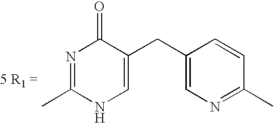

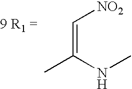

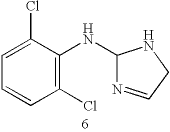

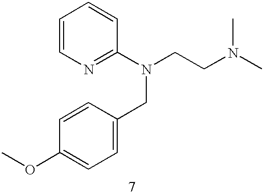

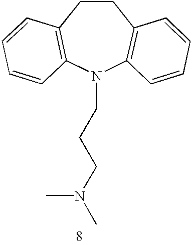

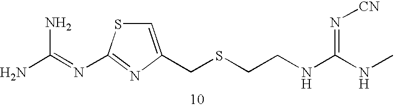

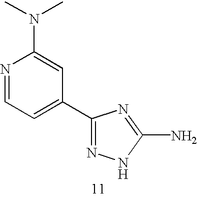

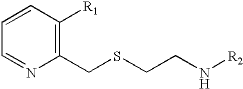

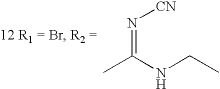

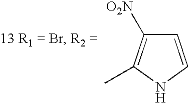

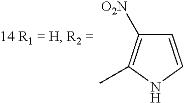

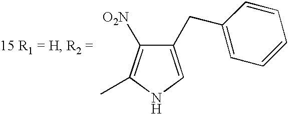

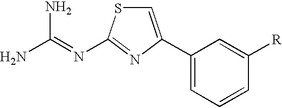

16 R = H
17 R = NH₂

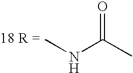

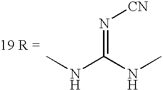

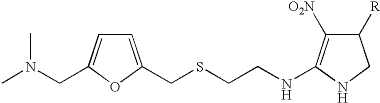

20 R = H

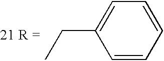

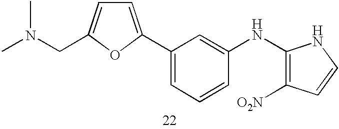

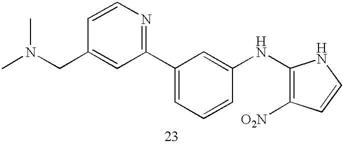

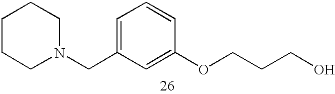

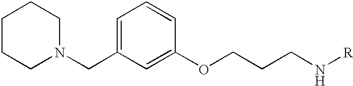

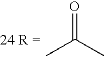

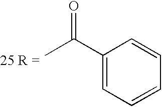

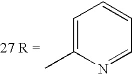

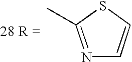

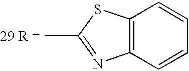

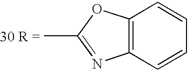

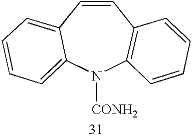

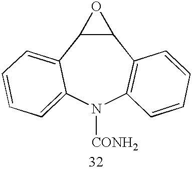

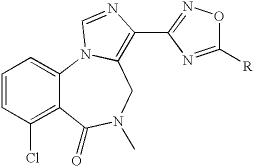

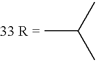

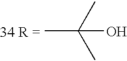

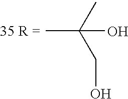

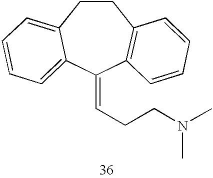

### Example 4

SPL macro (2dfp.spl) to prepare 2D-flngerprints for half life.

```
uims define macro 2dfp sybylbasic yes
##
## Set the Source Database, and Column-Names File.
##
setvar source %promptif(“$1” “STRING” “MYFILE.MDB” “Source Database.mdb”
“Database with molecules to be calculated”)
setvar resultsFP %promptif(“$ 1” “STRING” “Columns.txt” “Filename storing colunm
names” “Text file to store colunm names”)
## if %not(%mols(*))
##  %dialog_message(ERROR “There are no molecules.” “No Molecules”)
>$NULLDEV
##  return
## endif
#
# Set the molecule area to calculate “2D-FingerPrint”.
# Note that $current_molarea is defined by the “ calling”
# table when adding a column of data.
#
localvar mol_area
if $1
    setvar mol_area $1
else
    setvar mol_area $current_molarea
endif
database open $source read
##
## Loop over all molecules in the source database
##
for j IN %database(*)
    database get “$j” $mol_area
#
# set the SLN expression for the molecular area
#
    setvar sln_exp %s1n($mol_area)
    setvar ARRAY
#
## items + 1 (compd_num) BIT's will be used
######### @compdnum) ##########BIT 1
    setvar ARRAY[01] %mol_info($mol_area name)
####### Exp-Generator_read(file_ID) looping is another choice. . .
#############Unsaturated bonds ###### ###BIT 2˜4
## fp1a) Unsaturated bonds (aromatic)
    setvar query Any:Any
    setvar BIT %count(%search2D($sln_exp $query NoDuplicate 0 yes))
    setvar ARRAY[02] $BIT
## fb1b) Unsaturated bonds (bouble)
    setvar query Any=Any
    setvar BIT %count(%search2D($sln_exp $query NoDuplicate 0 yes))
    setvar ARRAY[03] $BIT
## fp1c) Unsaturated bonds (triple)
    setvar query Any#Any
    setvar BIT %count(%search2D($sln_exp $query NoDuplicate 0 yes))
    setvar ARRAY[04] $BIT
###############ring (topology) ############BIT 5˜15
## @fp2a) 3-membered ring
    setvar query Hev[1]˜Hev˜Hev@1
    setvar BIT %count(%search2D($sln_exp $query NoDuplicate 0 yes))
    setvar ARRAY[05] $BIT
## @fp2b) 4-membered ring
    setvar query Hev[1]˜Hev˜Hev˜Hev@1
    setvar BIT %count(%search2D($sln_exp $query NoDuplicate 0 yes))
    setvar ARRAY[06] $BIT
## @fp2c) 5-membered ring
    setvar query Hev[1]˜Hev˜Hev˜Hev˜Hev@1
    setvar BIT %count(%search2D($sln_exp $query NoDuplicate 0 yes))
    setvar ARRAY[07] $BIT
## @fp2d) 6-membered ring
    setvar query Hev[1 ]˜Hev˜Hev˜Hev˜Hev˜Hev@1
    setvar BIT %count(%search2D($sln_exp $query NoDuplicate 0 yes))
    setvar ARRAY[08] $BIT
## @fp2e) phenyl ring
    setvar query C[1]:C:C:C:C:C:@1
    setvar BIT %count(%search2D($sln_exp $query NoDuplicate 0 yes))
    setvar ARRAY[09] $BIT
## @fp2f) 7-membered ring
    setvar query Hev[1]˜Hev˜Hev˜Hev˜Hev˜Hev˜Hev@1
    setvar BIT %count(%search2D($sln_exp $query NoDuplicate 0 yes))
    setvar ARRAY[10] $BIT
## @fp2g) 8-membered ring
    setvar query Hev[1]˜Hev˜Hev˜Hev˜Hev˜Hev˜Hev˜Hev@1
    setvar BIT %count(%search2D($sln_exp $query NoDuplicate 0 yes))
    setvar ARRAY[11] $BIT
##@fp2h) 9-membered ring
    setvar query Hev[1]˜Hev˜Hev˜Hev˜Hev˜Hev˜Hev˜Hev˜Hev@1
    setvar BIT %count(%search2D($sln_exp $query NoDuplicate 0 yes))
    setvar ARRAY[12] $BIT
##@fp2i) 10-membered ring
    setvar query Hev[1]˜Hev˜Hev˜Hev˜Hev˜Hev˜Hev˜Hev˜Hev˜Hev@1
    setvar BIT %count(%search2D($sln_exp $query NoDuplicate 0 yes))
    setvar ARRAY[13] $BIT
##@fp2j) 11-membered ring
    setvar query Hev[1]˜Hev˜Hev˜Hev˜Hev˜Hev˜Hev˜Hev˜Hev˜Hev˜Hev@1
    setvar BIT %count(%search2D($sln_exp $query NoDuplicate 0 yes))
    setvar ARRAY[14] $BIT
##@fp2k) 12-membered ring
    setvar query
Hev[1]˜Hev˜Hev˜Hev˜Hev˜Hev˜Hev˜Hev˜Hev˜Hev˜Hev˜Hev@1
    setvar BIT %count(%search2D($sln_exp $query NoDuplicate 0 yes))
    setvar ARRAY[15] $BIT
################ Elements_Overall ############# BIT 16˜22
##@fp3a) total Hetro atoms
    setvar query Het
    setvar BIT %count(%search2D($snl_exp $query NoDuplicate 0 yes))
    setvar ARRAY[16] $BIT
##@fp3b) total Halogen
    setvar query Any[is=F,Br,Cl,I]
    setvar BIT %count(%search2D($sln_exp $query NoDuplicate 0 yes))
    setvar ARRAY[17] $BIT
##@fp3c) total N
    setvar query N
    setvar BIT %count(%search2D($sln_exp $query NoDuplicate 0 yes))
    setvar ARRAY[18] $BIT
##@fp3d) total NH
    setvar query NH
    setvar BIT %count(%search2D($sln_exp $query NoDuplicate 0 yes))
    setvar ARRAY[19] $BIT
##@fp3e) total O
    setvar query O
    setvar BIT %count(%search2D($sln_exp $query NoDuplicate 0 yes))
    setvar ARRAY[20] $BIT
##@fp3f) total OH
    setvar query OH
    setvar BIT %count(%search2D($sln_exp $query NoDuplicate 0 yes))
    setvar ARRAY[21] $BIT
##@fp3g) total S
    setvar query S
    setvar BIT %count(%search2D($sln_exp $query NoDuplicate 0 yes))
    setvar ARRAY[22] $BIT
################ Methyl, terminal ############# BIT 23˜26
##@fp4a) C-Methyl (omega-Oxidation)
    setvar query C—CH3
    setvar BIT %count(%search2D($sln_exp $query NoDuplicate 0 yes))
    setvar ARRAY[23] $BIT
##@fp4b) N-Methyl (N-demethylation)
    setvar query N—CH3
    setvar BIT %count(%search2D($sln_exp $query NoDuplicate 0 yes))
    setvar ARRAY[24] $BIT
##@fp4c) O-Methyl (O-demethylation)
    setvar query O—CH3
    setvar BIT %count(%search2D($sln_exp $query NoDuplicate 0 yes))
    setvar ARRAY[25] $BIT
##@fp4d) S-Methyl (S-demethylation)
    setvar query CH3—S[F]-Any[NOT=H*]
    setvar BIT %count(%search2D($sln_exp $query NoDuplicate 0 yes))
    setvar ARRAY[26] $BIT
################ Methylen —CH2— ############# BIT 27˜30
##@fp5a) Methylene group
    setvar query Any[NOT=H*,N,O]-CH2-Any[NOT=H*,N,O]
    setvar BIT %count(%search2D($sln_exp $query NoDuplicate 0 yes))
    setvar ARRAY[27] $BIT
##@fp5b) N-Methylene
    setvar query N—CH2-Any[NOT=H*]
    setvar BIT %count(%search2D($sln_exp $query NoDuplicate 0 yes))
    setvar ARRAY[28] $BIT
##@fp5c) O-Methylene
    setvar query O—CH2-Any[NOT=H*]
    setvar BIT %count(%search2D($sln_exp $query NoDuplicate 0 yes))
    setvar ARRAY[29] $BIT
##@fp5d) S-Methylene
    setvar query S[F]-CH2-Any[NOT=H*]
    setvar BIT %count(%search2D($sln_exp $query NoDuplicate 0 yes))
    setvar ARRAY[30] $BIT
################ Methine >CH—, Allylic/Benzylic H (to be absorded)
################ BIT 31˜36
##@fp6a) Methine group
setvar query Any[NOT=]H*,N,O,S]—CH(-Any[NOT=H*,N,O,S])-
Any[NOT=H*,N,O,S]
    setvar BIT %count(%search2D($sln_exp $query NoDuplicate 0 yes))
    setvar ARRAY[31] $BIT
##@fp6b) Benzylic H (Ar—CH) (if Ph—CH2, then the count =2)
    setvar query CHC(:Any):Any
    setvar BIT %count(%search2D($sln_exp $query NoDuplicate 0 yes))
    setvar ARRAY[32] $BIT
##@fp6c) Allyl H (if CR═CR—CH2, then the count =2)
    setvar query CHC(═C)
    setvar BIT %count(%search2D($sln_exp $query NoDuplicate 0 yes))
    setvar ARRAY[33] $BIT
##@fp6d) N-Methine
    setvar query N—CH(-Any[NOT=H*])-Any[NOT=H*]
    setvar BIT %count(%search2D($sln_exp $query NoDuplicate 0 yes))
    setvar ARRAY[34] $BIT
##@fp6e) O-Methine
    setvar query O—CH(-Any[NOT=H*])-Any[NOT=H*]
    setvar BIT %count(%search2D($sln_exp $query NoDuplicate 0 yes))
    setvar ARRAY[35] $BIT
##@fp6f) S-Methine
    setvar query O—CH(-Any[NOT=H*])-Any[NOT=H*]
    setvar BIT %count(%search2D($sln_exp $query NoDuplicate 0 yes))
    setvar ARRAY[36] $BIT
################ Nitrogen containing Compounds ############# BIT 37˜49
################ Amines/Imines/Nitrile ############# BIT 37˜46
##@fp7a) Primary Amines, unbranched
    setvar query NH2CH2
    setvar BIT %count(%search2D($sln_exp $query NoDuplicate 0 yes))
    setvar ARRAY[37] $BIT
##@fp7b) Primary Amines, branched
    setvar query NH2CH(Any[NOT=H*])(Any[NOT=H*])
    setvar BIT %count(%search2D($sln_exp $query NoDuplicate 0 yes))
    setvar ARRAY[38] $BIT
##@fp7c) Primary Amines, branched
    setvar query NH2C(Any[NOT=H*])(A0ny[NOT=H*])(Any[NOT=H*])
    setvar BIT %count(%search2D($sln_exp $query NoDuplicate 0 yes))
    setvar ARRAY[39] $BIT
##@fp7d) Primary Anilines (Ar—NH2)
    setvar query NH2C:Any(Any[NOT=H*])
    setvar BIT %count(%search2D($sln_exp $query NoDuplicate 0 yes))
    setvar ARRAY[40] $BIT
##@fp7e) Secondary Amines,
    setvar query NH(C[NOT=C=O])C[NOT═C═O]
    setvar BIT %count(%search2D($sln_exp $query NoDuplicate 0 yes))
    setvar ARRAY[41] $BIT
##@fp7f) Tertiary Amines
    setvar query N(C[NOT═C═O])(C[NOT═C═O])(C[NOT═C═O])
    setvar BIT %count(%search2D($sln_exp $query NoDuplicate 0 yes))
    setvar ARRAY[42] $BIT
##@fp7g) Imines
    setvar query N═C
    setvar BIT %count(%search2D($sln_exp $query NoDuplicate 0 yes))
    setvar ARRAY[43] $BIT
##@fp7h) Nitrile
    setvar query C#N
    setvar BIT %count(%search2D($sln_exp $query NoDuplicate 0 yes))
    setvar ARRAY[44] $BIT
##@fp7i) N in aromatics
    setvar query Any[is=N,C]:Any[is=N,C]
    setvar BIT %count(%search2D($sln_exp $query NoDuplicate 0 yes))
    setvar ARRAY[45] $BIT
##@fp7j) Guanidine
    setvar query NC(═N)N
    setvar BIT %count(%search2D($sln_exp $query NoDuplicate 0 yes))
    setvar ARRAY[46] $BIT
################ N˜N, Nitro, N—O ####################### BIT 47˜49
##@fp7k) NO {Hydroxyamine, Oxime, Hydroxamic acid, ....)
    setvar query N—O
    setvar BIT %count(%search2D($sln_exp $query NoDuplicate 0 yes))
    setvar ARRAY[47] $BIT
##@fp7l) Nitro (count =2), Nitroso (count =1)
    setvar query N(═O)
    setvar BIT %count(%search2D($sln_exp $query NoDuplicate 0 yes))
    setvar ARRAY[48] $BIT
##@fp7m) N˜N
    setvar query N˜N
    setvar BIT %count(%search2D($sln_exp $query NoDuplicate 0 yes))
    setvar ARRAY[48] $BIT
################ Amide, Ester, Sulfonamide ################ BIT 50˜52
##@fp8a) Ester
    setvar query C(═O)OC
    setvar BIT %count(%search2D($sln_exp $query NoDuplicate 0 yes))
    setvar ARRAY[50] $BIT
##@fp8b) Amide
    setvar query NC(═O)
    setvar BIT %count(%search2D($sln_exp $query NoDuplicate 0 yes))
    setvar ARRAY[51] $BIT
##@fp8c) Sulfonamide
    setvar query NS(═O)(═O)
    setvar BIT %count(%search2D($sln_exp $query NoDuplicate 0 yes))
    setvar ARRAY[52] $BIT
################ Ketone, Aldehyde, Alcohol, Thiol, Sulfide ### BIT 53˜59
##@fp9a) Primary Alcohol
    setvar query CH2(OH)(˜Hev)
    setvar BIT %count(%search2D($sln_exp $query NoDuplicate 0 yes))
    setvar ARRAY[53] $BIT
##@fp9b) Secondary Alcohol
    setvar query CH(OH)(˜Hev)(˜Hev)
    setvar BIT %count(%search2D($sln_exp $query NoDuplicate 0 yes))
    setvar ARRAY[54] $BIT
##@fp9c) Ketone, Aldehyde
    setvar query Any[is=H,Cl]CC(═O)(Any[is=H,C])
    setvar BIT %count(%search2D($sln_exp $query NoDuplicate 0 yes))
    setvar ARRAY[55] $BIT
##@fp9d) COOH
    setvar query Any[is=H,C]CC(═O)(OH)
    setvar BIT %count(%search2D($sln_exp $query NoDuplicate 0 yes))
    setvar ARRAY[56] $BIT
##@fp9e) Sulfide
    setvar query CS[F]C
    setvar BIT %count(%search2D($sln_exp $query NoDuplicate 0 yes))
    setvar ARRAY[57] $BIT
##@fp9f) Thiol
    setvar query S[F]H(C)
    setvar BIT %count(%search2D($sln_exp $query NoDuplicate 0 yes))
    setvar ARRAY[58] $BIT
##@fp9g) Thiocarbonyl
    setvar query C═S
    setvar BIT %count(%search2D($sln_exp $query NoDuplicate 0 yes))
    setvar ARRAY[59] $BIT
    echo $ARRAY
    echo $ARRAY >> $resultsFP
    zap $mol_area
endfor
database close
##
## Announce of the completion & the location of the results
echo
echo “job completed on %system(date)”
echo “The results is stored $resultsFP as a text file,”
echo “please import if from MSS (table).”
echo “ ═>custom format, space separated, column lable used”
```

### Example 5

By use of a similar method described in example 4, 2D-Fingerprints for Caco-2 permeability and blood-brain barrier partition were prepared based on the following description.

| | | |
|---|---|---|
| Fp-ID | Name | Query |

Alky Amines

| | | |
|---|---|---|
| fp1a | Primary | NH2C[NOT=C═O,C═S,C:Any,Any[IS=C,N]C(═N)N,C[1](Any[IS=O,S,N] |
| | | Any=:AnyAny=@1),C[1](=AnyAny[IS=O,S,N]Any=:Any@1)] |
| fp1b | Secondary | NH(C[NOT=C═O,C═S,C:Any,Any[IS=C,N]C(═N)N,C[1](Any[IS=O,S,N]A |
| | | ny=:AnyAny=@1),C[1](=AnyAny[IS=O,S,N]Any=:Any@1)])(C[NOT=C═ |
| | | O,C═S,C:Any,Any[IS=C,N]C(═N)N,C[1](Any[IS=O,S,N]Any=:AnyAny= |
| | | @1),C[1](=AnyAny[IS=O,S,N]Any=:Any@1)]) |
| fp1c | Tertiary | N(C[NOT=C═O,C═S,C:Any,Any[IS=C,N]C(═N)N,C[1](Any[IS=O,S,N]An |
| | | y=:AnyAny=@1),C[1](=AnyAny[IS=O,S,N]Any=:Any@1)])(C[NOT=C═O |
| | | ,C═S,C:Any,Any[IS=C,N]C(═N)N,C[1](Any[IS=O,S,N]Any=:AnyAny=@1 |
| | | ),C[1](=AnyAny[IS=O,S,N]Any=:Any@1)])(C[NOT=C═O,C═S,C:Any,Any |
| | | [IS=C,N]C(═N)N,C[1](Any[IS=O,S,N]Any=:AnyAny=@1),C[1](=AnyAny[ |
| | | IS=O,S,N]Any=:Any@1)]) |

Amines attached to heteroaromatics

| | | |
|---|---|---|
| fp2a | Primary | C[1](Any[IS=O,S,N]Any=:AnyAny=@1)NH2 |
| | | C[1](=AnyAny[IS=O,S,N]Any=:Any@1)NH2 |
| fp2b | Secondary | C[1](Any[IS=O,S,N]Any=:AnyAny=@1)NHAny[NOT=H*] |
| | | C[1](=AnyAny[IS=O,S,N]Any=:Any@1)NHAny[NOT=H*] |
| fp2c | Tertiary | C[1](Any[IS=O,S,N]Any=:AnyAny=@1)N(Any[NOT=H*])Any[NOT=H*] |
| | | C[1](=AnyAny[IS=O,S,N]Any=:Any@1)N(Any[NOT=H*])Any[NOT=H*] |

Anilines

| | | |
|---|---|---|
| fp3a | Primary | NH2C(:Any)(:Any[NOT=H*]) |
| fp3b | Secondary | NH(C(:Any)(:Any[NOT=H*]))Any[NOT=H*] |
| fp3c | Tertiary | N(C(:Any)(:Any[NOT=H*]))(Any[NOT=H*])Any[NOT=H*] |
| | | N(C(:Any)(:Any[NOT=H*]))═C |

N in aromatics

| | | |
|---|---|---|
| fp4a | 6-membered | Any[is=N,C]:N:Any[is═N,C] |
| | ring | |
| fp4b | —NH— in | N[1]HAny[IS=C,N]:Any[IS=C,N]Any[IS=C,N]═:Any[IS=C,N]-@1 |
| | heteroaromatics | |
| fp4c | —N— in | N[1]Any[IS=C,N]═:Any[IS=C,N]Any[IS=C,N]═:Any[IS=C,N]-@1 |
| | heteroaromatics | |
| fp4d | —N═ in | N[1](Any[IS=O,S,N]Any=:AnyAny=@1) |
| | heteroaromatics | N[1](=AnyAny[IS=O,S,N]Any=:Any@1) |

Imines/Nitrile/Guanidine/Amidine

| | | |
|---|---|---|
| fp5a | Imines | Any[IS=C,H,S]N[NOT=N[1](Any[IS=O,S,N]Any=:AnyAny@1),N[1](=A |
| | | nyAny[IS=O,S,N]Any=:Any=@1)]═C[NOT=Any[IS=C,N]C(═N)N] |
| fp5b | Nitrile | C#N |
| fp5c | Guanidine | N[NOT=C[1](Any[IS=O,S,N]Any=:AnyAny=@1)N,C[1](=AnyAny[IS=O,S |
| | | ,N]Any=:Any@1)N]C(═N)N[NOT=C[1](Any[IS=O,S,N]Any=:AnyAny=@ |
| | | 1)N,C[1](=AnyAny[IS=O,S,N]Any=:Any@1)N] |
| fp5d | Amidine | Any[NOT=N]C(═N[NOT=N[1](Any[IS=O,S,N]Any=:AnyAny=@1),N[1](= |
| | (not hetero- | AnyAny[IS=O,S,N]Any=:Any@1)])N |
| | aromatics) | |

N˜O/Nitro/N═N/N—N

| | | |
|---|---|---|
| fp6a | Hydroxyamine, | N[!r]-O[!r] |
| | Oxime, | |
| | Hydroxamic | |
| | acid . . . ) | |
| fp6b | Nitro, Nitroso | N(═O) |
| fp6c | N═N Azo (not | N═N[NOT=N[1](Any[IS=O,S,N]Any=:AnyAny=@1),N[1](=AnyAny[IS=O |
| | in a ring) | ,S,N]Any=:Any@1)] |
| fp6d | N—N Hydrazine | N—N[NOT=N[1](Any[IS=O,S,N]Any=:AnyAny@1),N[1](=AnyAny |
| | | [IS=O,S,N]Any=:Any@1)] |

Amide/Thioamide/Sulfonamide

| | | |
|---|---|---|
| fp7a | Amide1 | NH2C═O |
| | (NH₂—CO) | |
| fp7b | Amide2 | Any[NOT=H*]NHC═O |
| | (R₁—NH—CO) | |
| fp7c | Amide3 | Any[NOT=H*]N(C═O) Any[NOT=H*] |
| | (R₁R₂N—CO) | |
| fp7d | Thioamide1 | NH2C═S |
| | (NH₂—CS) | |
| fp7e | Thioamide2 | Any[NOT=H*]NHC═S |
| | (R₁—NH—CS) | |
| fp7f | Thioamide3 | Any[NOT=H*]N(C═S)Any[NOT=H*] |
| | (R₁R₂N—CS) | |
| fp7g | Sulf.amide1 | NH2S(═O)(═O) |
| | (NH₂SO₂) | |
| fp7h | Sulf.amide2 | NH(S(═O)═O)Any[NOT=H*] |
| | (R₁—NHSO₂) | |
| fp7i | Sulf.amide3 | N(S(═O)═O)(Any[NOT=H*])Any[NOT=H*] |
| | (R₁R₂—NSO₂) | |

Alcohol/Ether/Aldehyde/Ketone/Ester/Carboxylic acid/Carbothioic
acid/Sulfinic acid/Sulfonic acid

| | | |
|---|---|---|
| fp8a | Alcohol | C[NOT=C═O,C═S](OH) |
| fp8b | Ether | Any[NOT=C═O, H*]-O-Any[NOT=C═O,H*] |
| fp8c | Aldehyde | CCH(═O) |
| fp8d | Ketone | CC(═O)C |
| fp8e | Ester | C(═O)OC |
| fp8f | Carboxylic acid | C(═O)(OH) |
| fp8g | Carbothioic O | C(═S)(OH) |
| | acid | |
| fp8h | Carbothioic S | C(═O)(SH) |
| | acid | |
| fp8i | sulfinic acid | Any[is═H,C]S[NOT=S(═O)(═O)](═O)(OH) |
| fp8j | sulfonic acid | Any[is═H,C]S(═O)(═O)(OH) |

Halogen

| | | |
|---|---|---|
| fp9a | Fluoro | F |
| fp9b | Chloro | Cl |
| fp9c | Bromo | Br |
| fp9d | Iodo | I |

Total C/H/N/O/S

| | | |
|---|---|---|
| fp10a | total C | C |
| fp10b | total H | H |
| fp10c | total N | N |
| fp10d | total O | O |
| fp10e | total S | S |

1. A method for predicting pharmacokinetic properties of molecules comprising the steps of:
(a) preparing 2D-structures of molecules used as a training set;
(b) constructing a 2D-fingerprint by counting the number of structural descriptors that potentially relate to a pharmacokinetic property, either manually or automatically using internally developed macro; wherein said structural descriptors consist of predefined 20 to 80 atoms/fragments or substructures;
(c) analyzing the obtained 2D-fingerprint by a statistical analysis method to correlate with the pharmacokinetic property of the molecule to yield a quantitative structure-property relationship (QSPR) model; and
(d) calculating the pharmacokinetic property of a trial molecule using the above obtained QSPR model.

2. A method of claim 1, wherein the pharmacokinetic property is absorption.

3. A method of claim 1, wherein the pharmacokinetic property is distribution.

4. A method of claim 1, wherein the pharmacokinetic property is metabolism

5. A method of claim 1, wherein the pharmacokinetic property is excretion.

6. A method of claim 1, wherein the internally developed macro comprises the macro script 2dfp.spl or 2dfp_abs.spl, written in SYBYL™ Programming Language (SPL).

7. A system for predicting pharmacokinetic properties of molecules comprising:
(a) means for preparing 2D-structures of molecules used as a training set;
(b) means for constructing a 2D-fingerprint by counting the number of structural descriptors that potentially relate to a pharmacokinetic property, wherein said structural descriptors consist of predefined 20 to 80 atoms/fragments or substructures;
(c) means for analyzing the obtained 2D-fingerprint by a statistical analysis method to correlate with the pharmacokinetic property of the molecule to yield a quantitative structure-property relationship (QSPR) model; and
(d) means for calculating the pharmacokinetic property of a trial molecule using the above obtained QSPR model.
